(19) United States
(12) Patent Application Publication
(10) Pub. No.: US 2015/0177221 A1
(43) Pub. Date: Jun. 25, 2015

(54) SIMULATED CONTAMINATED SKIN FORMULATION

(71) Applicant: L'Oreal, Paris (FR)

(72) Inventor: Gregory Calvin Peterson, Seattle, WA (US)

(73) Assignee: L'Oreal, Paris (FR)

(21) Appl. No.: 14/135,549

(22) Filed: Dec. 19, 2013

(51) Int. Cl. G01N 33/483; G01N 21/27

(52) U.S. Cl. CPC G01N 33/4833; G01N 21/27

(57) ABSTRACT

Disclosed herein are formulations configured to simulate “dirty” skin that has been contaminated with particulate pollution, natural skin secretions, and applied product (e.g., cosmetics). Relatedly, also disclosed are methods for determining the efficacy of a cleansing procedure that include applying the formulation to clean skin, cleaning the formulation from the skin, and determining the amount of formulation remaining on the skin. The relative amounts of formulation at each stage can be determined, for example, using colorimetric or digital image analyses.

### BACKGROUND

Pollution is grossly defined as the presence or introduction into the environment of a substance or thing that has harmful or poisonous effects. Pollutants consist of a range of biological, chemical, and mineral substances generated from industrial, agricultural, and municipal development to name a few. The World Health Organization (WHO) defines air pollution as contamination of indoor or outdoor environments by any chemical, physical, or biological agent that modifies the natural characteristics of the atmosphere. Household combustion devices, motor vehicles, industrial facilities, forest fires, and natural disturbances (e.g., volcanic eruptions) are common sources of air pollution. Atmospheric pollutants of major public health concern include particulate matter, carbon monoxide, ozone, nitrogen dioxide, and sulfur dioxide. The U.S. Environmental Protection Agency (EPA) and the WHO have summarized the global extent of common atmospheric pollutants as well as the hundreds of studies that have investigated its impact on environmental and human health. Health effects most commonly recognized of critical concern include those associated with respiratory, cardiovascular, and neurological disease as well as allergies and cancer.

The link of atmospheric pollution to skin aging is of increasing concern worldwide, with new evidence of a cause-effect relationship emerging. Particulate pollution is described in terms of particle size: PM_{2.5} and PM₁₀ having an aerodynamic diameter less than 2.5 mm and 10 mm, respectively. Carbon black (soot) and dust (mineral oxides, such as iron oxides and the like) comprise much of the particulate matter in these size ranges. Researchers have recently assessed the association of pigmented spots on the forehead and cheeks with pollution. An increase in soot and particles from traffic were associated with 20% more pigment spots on the forehead and cheeks than those in more rural communities having lower atmospheric carbon levels. The number of spots increases to 35% for those living within 100 m of high traffic areas.

While the link between skin aging and particulate pollution is in early stages of investigation, the consumer-perceived link has emerged as a significant concern for many living in urban communities with high atmospheric pollution. In order to assess the cleansing efficacy of a skincare brush and various cleansers on the removal of atmospheric pollution, the importance of a real-life surrogate of dirty skin becomes paramount. Dirty skin is not only the accumulation of soot and dust onto the skin's surface but comprises natural skin byproducts: decomposition products from cornification (cellular debris and lipids), sebaceous secretions, microorganisms and their byproducts, as well as products intentionally or incidentally applied to the skin (e.g., moisturizers, sunscreens, makeup, grease, etc.). All of these components attract and retain environmental dirt and pollutants. Various surrogate models exist for dirty skin and pollutants; however no surrogate model exists which combines the core components of sebum, common atmospheric pollutants (defined particulate matter below common standards of PM_{2.5}, PM₁₀,) and components incidentally or intentionally applied to the skin.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a formulation configured to simulate contaminated human skin is provided. In one embodiment, the formulation includes:

a synthetic sebum composition, comprising:
one or more triglycerides;
one or more fatty acids;
one or more waxes or wax esters;
one or more hydrocarbons; and
cholesterol or a cholesterol ester; and

a plurality of particles, wherein the plurality of particles has a mean diameter of 10 microns or less.

In another aspect, a preferred formulation configured to simulate contaminated human skin is provided that includes:

a synthetic sebum composition, comprising:
cholesterol;
squalane;
oleic acid;
glyceryl trioleate; and
cetyl palmitate; and

a plurality of particles, wherein the plurality of particles has a mean diameter of 10 microns or less.

In another aspect, a formulation is provided that consists essentially of cholesterol; squalane; oleic acid; glyceryl trioleate; cetyl palmitate; and a plurality of particles, wherein the plurality of particles have a mean diameter of 10 microns or less.

In another aspect, a method of evaluating the efficacy of a skin-cleansing procedure is provided. In one embodiment, the method includes:

applying a formulation as disclosed herein to a test area of a subject's skin;

performing a skin-cleansing procedure on the test area;

measuring the color of the test area to provide a post-clean color signal; and

comparing the post-clean color signal to a baseline color signal obtained without the formulation.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 illustrates a method of evaluating the efficacy of a skin-cleansing procedure in accordance with the disclosed embodiments;

FIG. 2 graphically illustrates the correlation between color intensity and relative concentration of carbon black in an exemplary formulation in accordance with the disclosed embodiments;

FIG. 3 is a chart comparing the amount of cleansing efficacy of a sonic brush compared to manual cleansing; and

FIG. 4 is a chart comparing the cleansing efficacy of a sonic brush at different brush speeds.

### DETAILED DESCRIPTION

Disclosed herein are formulations configured to simulate “dirty” skin that has been contaminated with particulate pollution, natural skin secretions, and applied product (e.g., cosmetics). Relatedly, also disclosed are methods for determining the efficacy of a cleansing procedure that include applying the formulation to clean skin, cleaning the formulation from the skin, and determining the amount of formulation remaining on the skin. The relative amounts of formulation at each stage can be determined, for example, using colorimetric or digital image analyses.

The formulation includes key components of sebum (e.g., squalane, triglycerides, fatty acids, wax esters, cholesterol); applied product (natural and synthetic wax esters and oil); and particulate pollutants (e.g., iron oxides, carbon black) in an effort to mimic the conditions experienced by skin exposed to the modern urban environment. The formulation is sufficiently robust in its adherence to the skin to mimic dirty skin and aid in evaluation of the cleansing efficacy and synergies between cleansing devices and cleansing formulations. The formulation can be manufactured using common cosmetic ingredients.

As will be described in greater detail below, the evaluation tests are non-invasive tests using optical analysis (e.g., digital photographs), dermatological instruments, and image analysis software to quantify the cleansing efficacy of various cleansing devices, cleansers, and/or their combination.

In one aspect, a formulation configured to simulate contaminated human skin is provided. In one embodiment, the formulation includes:

a synthetic sebum composition, comprising:
one or more triglycerides;
one or more fatty acids;
one or more waxes or wax esters;
one or more hydrocarbons; and
cholesterol or a cholesterol ester; and

a plurality of particles, wherein the plurality of particles has a mean diameter of 10 microns or less.

### Synthetic Sebum

The synthetic sebum composition is formulated to mimic the film covering typical human skin. The triglycerides, fatty acids, waxes (and/or wax esters), and cholesterol (or cholesterol ester) are configured to mimic natural skin secretions. The wax esters and hydrocarbons can be configured to mimic typical applied skin product (e.g., cosmetics).

In one embodiment, the one or more triglycerides are selected from the group consisting of glyceryl trioleate, triglyceride-rich oils (e.g., olive oil, cotton seed oil, coconut oil), and combinations thereof. In one embodiment, the one or more triglycerides are present in an amount from 15 to 60% by weight.

In one embodiment, the one or more fatty acids are selected from the group consisting of oleic acid, palmitic acid, palmitoleic acid, and combinations thereof. In one embodiment, the one or more fatty acids are present in an amount from 10 to 35% by weight.

In one embodiment, the one or more waxes or wax esters are selected from the group consisting of cetyl palmitate, paraffin wax, spermaceti wax, beeswax, jojoba oil, lanolin, and combinations thereof. In one embodiment, the one or more waxes or wax esters are present in an amount from 15 to 35% by weight.

In one embodiment, the one or more hydrocarbons are selected from the group consisting of squalene, squalane, petrolatum, mineral oil, and combinations thereof. In one embodiment, the one or more hydrocarbons are present in an amount from 10 to 35% by weight.

In one embodiment, the formulation further comprises an emulsifier (e.g., polyglyceryl oleate). In one embodiment, the emulsifier is present in an amount of from 0.01 to 4% by weight.

### Particulate

In the formulation, particulate matter is added to the synthetic sebum composition to create a robust (i.e., difficult to remove) surrogate for dirty, polluted skin to more effectively evaluate differences between devices, cleansers, their combination, as well as other methods of cleansing the skin.

This methodology was developed to evaluate the cleansing efficacy of cosmetic cleansing devices, cosmetic cleansers, and/or their combination in their ability to remove common skin pollutants/atmospheric particulate matter. The direct application of particulate matter to the skin's surface in order to assess cleansing efficacy has limited utility. In real-life situations, pollutants/atmospheric particulate matter is trapped in grease/oil typical of human sebum and in products applied to the skin directly or indirectly (makeup, grease from handling food, as well as many items used daily). This combination of natural and environmental grease and oils combined with the accumulation of atmospheric pollution from fuel combustion and dust accumulate on the skin's surface and within the skin's pores resulting in material that is very difficult to remove with superficial cleansing/hygiene regimes.

The formulation comprises particles configured to mimic airborne particulate pollution of the type found in urban environments. Such particles can become trapped in the sebum of the skin and can negatively affect skin health and appearance. By including such particles, the formulation more accurately mimics the actual skin conditions experienced by urban inhabitants. The particles have a mean diameter of 10 microns or less, so as to be consistent with the diameter of real world pollutants of the type mimicked by the formulation.

“Particulate matter,” also known as particle pollution or PM, is a complex mixture of extremely small particles and liquid droplets. Particle pollution is made up of a number of components, including acids (such as nitrates and sulfates), organic chemicals, metals, and soil or dust particles. Particulate pollution is commonly measured in terms of PM 2.5 (particulates measuring 2.5 microns in diameter or less) or PM 10 (particulates measuring 10 microns in diameter or less).

Particulate matter that generally causes visual effects such as smog consists of sulfur dioxide, nitrogen oxides, carbon monoxide, mineral dust (e.g., iron oxides), organic matter, and elemental carbon (also known as black carbon or soot).

The size of particles is directly linked to their potential for causing health problems. The EPA is concerned about particles that are 10 micrometers in diameter or smaller because those are the particles that generally pass through the throat and nose and enter the lungs. Once inhaled, these particles can affect the heart and lungs and cause serious health effects. EPA groups particle pollution into two categories:

“Inhalable coarse particles,” such as those found near roadways and dusty industries, are larger than 2.5 micrometers and smaller than 10 micrometers in diameter. Representative coarse particles include silica, silicon dioxide (SiO2), aluminum oxide (Al2O3), iron oxide(s) (FeO, Fe2O3), calcium oxide (CaO), and carbonates (CaCO3, mgCO3).

“Fine particles,” such as those found in smoke and haze, are 2.5 micrometers in diameter and smaller. These particles can be directly emitted from sources such as forest fires, or they can form when gases emitted from power plants, industries, and automobiles react in the air. Representative fine particles in the formulation include carbon black, vegetable carbon, and lamp black, all of which are sources of elemental carbon. In a preferred embodiment, the formulation includes carbon black that is 20 to 60 nanometers in diameter with a mean diameter of 40 nanometers. In one embodiment, the fine particles in the formulation have a mean diameter of 500 nanometers or less.

The formulation comprises inhalable coarse particles as well as fine particles in order to simulate both types of particle pollution.

In certain embodiments, darkly pigmented substances, such as particulate cosmetic colorants, are used as surrogates for particulate atmospheric pollution. In one embodiment, the plurality of particles is selected from the group consisting of carbon black particles, iron oxide particles, and combinations thereof. Carbon black particles mimic soot and iron oxide particles mimic dust, two of the most common particulate pollutants.

In one preferred embodiment, the plurality of particles comprises carbon black particles having a mean diameter of about 500 nanometers or less and iron oxide particles having a mean diameter of about 0.3 to about 5 microns. These size ranges are typical of actual particulate pollution of the type mimicked by the formulation.

In another aspect, a preferred formulation configured to simulate contaminated human skin is provided that includes:

a synthetic sebum composition, comprising:
cholesterol;
squalane;
oleic acid;
glyceryl trioleate; and
cetyl palmitate; and

a plurality of particles, wherein the plurality of particles has a mean diameter of 10 microns or less. This preferred composition has been demonstrated experimentally to accurately mimic dirty skin in the manner desired. As disclosed below, the preferred formulation has been used to successfully test the efficacy of various cleaning methods.

In one embodiment, cholesterol is present in an amount from 2 wt % to 6 wt %.

In one embodiment, squalane is present in an amount from 5 wt % to 30 wt %.

In one embodiment, oleic acid is present in an amount from 10 wt % to 35 wt %.

In one embodiment, glyceryl trioleate is present in an amount from 15 wt % to 60 wt %.

In one embodiment, cetyl palmitate is present in an amount from 15 wt % to 35 wt %.

In one embodiment, the plurality of particles comprises carbon black present in an amount from 0.05 wt % to 4 wt %.

In one embodiment, the plurality of particles comprises iron oxides present in an amount from 0.05 wt % to 4 wt %.

In yet another aspect, a formulation is provided that consists essentially of cholesterol; squalane; oleic acid; glyceryl trioleate; cetyl palmitate; and a plurality of particles, wherein the plurality of particles has a mean diameter of 10 microns or less.

### Formulation Mixing

The disclosed formulations can be formed in any manner known to those of skill in the art. Mixing the components is a typical technique used. As disclosed in the Exemplary Formulation below, the components may be mixed in different phases. Phased mixing allows particular components to be isolated or combined in a manner that provides the most efficient mixing.

### Cleansing Efficacy Testing Methods

In another aspect, a method of evaluating the efficacy of a skin-cleansing procedure is provided. In one embodiment, the method includes:

applying a formulation as disclosed herein to a test area of a subject's skin;

performing a skin-cleansing procedure on the test area;

measuring the color of the test area to provide a post-clean color signal; and

comparing the post-clean color signal to a baseline color signal obtained without the formulation.

In this aspect, the anti-pollution/cleansing effect of a cosmetic cleansing product is evaluated by colorimetric and/or image analysis to quantify the amount of darkly pigmented (particulate matter) from the formulation removed and/or remaining on the skin after cleansing.

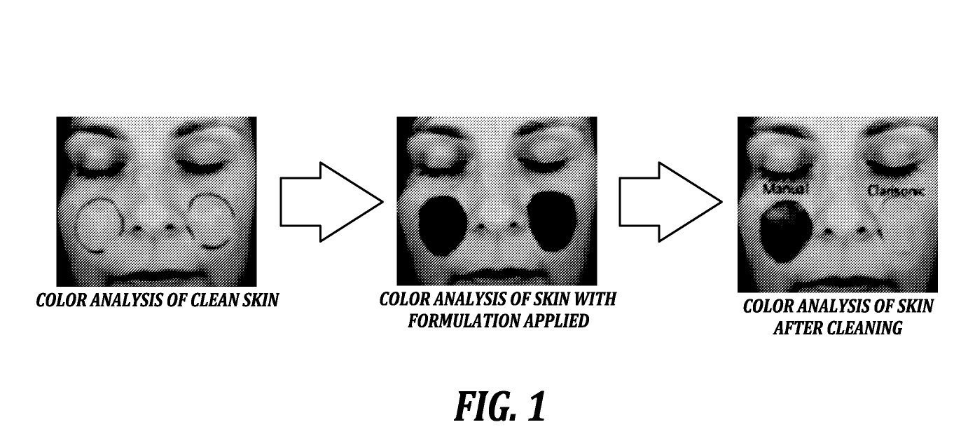

The method may be better understood with reference to FIG. 1, which illustrates a typical process flow. First, the skin is prepared for application of the formulation (e.g., by washing) and color analysis is performed on the test area. Color analysis may be colorimetric analysis, digital image analysis, or the like. The “clean skin” color analysis provides a baseline color signal.

Next, the formulation is applied to the skin (e.g., by hand or with an applicator). Color analysis is again performed to provide a pre-clean color signal.

Cleansing is then performed on the formulation/skin. Any cleansing method can be used. In the exemplary test illustrated in FIG. 1, a side-by-side test comparing manual cleansing (e.g., by hand) to cleansing with a Clarisonic sonic brush is pictured. A final color analysis is performed to provide a post-clean color signal.

The baseline color signal, pre-clean color signal, and post-clean color signal are used to evaluate the efficacy of the cleansing of the skin by determining the amount of formulation remaining on the skin after cleansing. The less formulation remaining on the skin, the more effective the cleansing method is determined to be.

The amount of formulation removed is calculated from the difference pre-cleansing [post-makeup application skin color between baseline (untreated) skin color] to post-cleansing [post-cleansing skin color minus baseline (untreated) skin color]. The amount of makeup remaining is calculated from the differencing in post-cleansing skin color compared to baseline (untreated) skin color.

In one embodiment, the baseline color signal is obtained in the test area prior to the step of applying the formulation. As illustrated in FIG. 1, the baseline color signal can be obtained prior to applying the formulation. However, it will be appreciated that the baseline color signal can be obtained post-cleansing and after thorough washing of the skin to remove all formulation. Furthermore, a subject's baseline color signal could be kept on record (e.g., digitally) and the method performed without obtaining a baseline color signal at the same sitting as obtaining the pre-clean and post-clean color signals.

In one embodiment, the step of measuring the color of the test area comprises digital photograph analysis. The non-invasive and common nature of digital photography and subsequent image analysis provides many benefits to the disclosed methods. While digital photography is preferred, colorimetric analysis and any other technique known to those of skill in the art as being capable of quantitatively determining color on a surface (e.g., spectrometer/spectrophotometer) are compatible with the disclosed methods.

In one embodiment, the test area is on the subject's face or forearm. The face and forearms are preferred locations for testing due to their ease of access and their frequent exposure to atmospheric pollution, which makes them skin areas of interest. Any skin area of the body can be used, however.

In one embodiment, the skin-cleansing procedure is selected from the group consisting of manual cleansing, cleansing with a rotating brush, and cleansing with an oscillating brush. Manual cleansing may include cleansing cloths, pads, sponge, wipes, puffs, and brushes. Oscillating brushes include sonic brushes such as the Clarisonic brushes.

In one embodiment, the formulation is applied to the test area in an amount of about 1 to about 10 mg/cm². Other amounts of formulation can be used, as needed to mimic particular levels of pollution.

### Exemplary Formulation

Formulation Ingredients

| Component | Grams | Phase |
|---|---|---|
| Polyglyceryl Oleate | 2.5 | A |
| Cholesterol | 3.5 | A |
| Squalane | 7 | A |
| Oleic Acid | 15 | A |
| Glyceryl Trioleate | 15 | A |
| Cetyl Palmitate | 8 | A |
| Mineral Oil | 10 | A |
| Petrolatum | 10 | A |
| Water | 20 | B |
| Carbon Black (CI77266) | 4 | B |
| Iron Oxides - Brown | 4 | B |
| Paraben-DU | 1 | C |
| Total | 100 | |

The exemplary formulation is mixed as follows:
i. Add phase A into a disinfected, heat-resistant glass beaker and heat to 150° F. to melt the ingredients.
ii. Heat phase B in a separate beaker. Once phase B has melted, stir well and slowly add phase A ingredients into phase B. Remove from heat, stirring well (homogenizing) until a homogenous cream is formed.
iii. After the temperature has dropped below 100° F., add phase C, mix well. Fill jars.

### Exemplary Methods of Analysis

Test Methodology and Equipment

Testing can be on any skin surface. Exemplary skin surfaces include the face (cheeks) and arms (forearms). Certain cameras and systems are preferable for each body part.

Camera Specifications:
Face: VISIA CR (Canfield Imaging Systems), Standard II photograph.
Arms: Canon EOS 5D Mark II with light stand.

Image Analysis

Digital images are analyzed as follows. Image J (NIH; Bethesda, Md.) is used to analyze the number of white or black pixels (0=black, 255=white) within the area of the circles at baseline, pre-cleansing, and post-cleansing.

Statistical Analysis

Once the image analysis is complete, the resulting data is separated into treatment groups (e.g., Clarisonic brush versus manual cleansing). The normality of differences (Baseline—post-clean) is determined using the Shapiro-Wilk test. Parametric paired t-tests are performed. Multiple treatment groups can be analyzed using analysis of variance (ANOVA). Note: For normality tests, the statistical significance level was set at p≦0.01. For the paired t-test, the statistical significance level was set at p≦0.05.

Cleansing Protocol

An exemplary facial testing protocol is as follows:
i. Subject photograph is taken.
ii. Formulation is applied to each treatment area (approximately 3 μg/cm2) and spread evenly.
iii. Subject photograph is taken.
iv. Per randomization schedule, the esthetician identifies which cheek will be cleansed with which cleansing method on the right and left cheeks.
v. The right cheek is always cleansed first.
vi. The right cheek is misted once for 2 seconds with water from a bottle of facial spray (e.g., Evian Facial Spray), held approximately 6 inches from the face (˜0.6 mL of water on the cheek area which is ˜7 cm in diameter).
vii. 0.5 cc of cleanser for the right cheek is measured using a syringe and applied to the center of the circle to be cleansed.
viii. The esthetician dampens the brush head by placing in water (shaking off the excess); or for manual cleansing, the esthetician dampens index, middle, and ring fingers.
ix. The esthetician cleanses the designated area on the right cheek for seconds (per cleansing method identified in the randomization schedule).
x. Using 2 damp pieces of gauze, the study esthetician gently blots the cleansed area twice to remove excess cleanser.
xi. The esthetician repeats the same steps on the left cheek using the other method of cleansing per the randomization schedule. Front view image is taken (day light), VISIA-CR.

An exemplary forearm testing protocol is as follows:
i. Three circles, each with a 1.25-inch diameter, are traced on the volar surface of both forearms using a template. The circles will be placed centrally on the forearms (at least 2 cm from the wrist joint and at least 2 cm from the elbow joint) and adjacent test sites will be separated by ≧2 cm. The circles will be labeled as each test site.
ii. Images are taken with the Canon EOS 5D Mark II with light stand.
iii. Formulation is applied to each treatment area (approximately 3 μg/cm2) and spread evenly.
iv. Per randomization schedule, the esthetician identifies which test site will be cleansed with which cleanser/cleansing method. If skin should be cleansed wet, test site 1 is misted once for 2 seconds with water from a bottle of Evian Facial Spray at ˜6 inches from the test site.
v. 0.25 cc (using a syringe) or a “pinch” measuring spoon (more viscous formulations ˜0.25 cc) of the cleanser for test site 1 is measured and applied to the center of the circle to be cleansed.
vi. The esthetician cleanses test site 1 per cleanser/cleansing method identified in the randomization schedule for 5 seconds (using timer).
vii. Using 2 damp pieces of gauze, the study esthetician gently blots the cleansed area twice to remove excess cleanser.
viii. Cleansing is repeated as above for each test site.
ix. Images are taken with the Canon EOS 5D Mark II with light stand.

### Correlation of Color Intensity and Particulate Matter Concentration

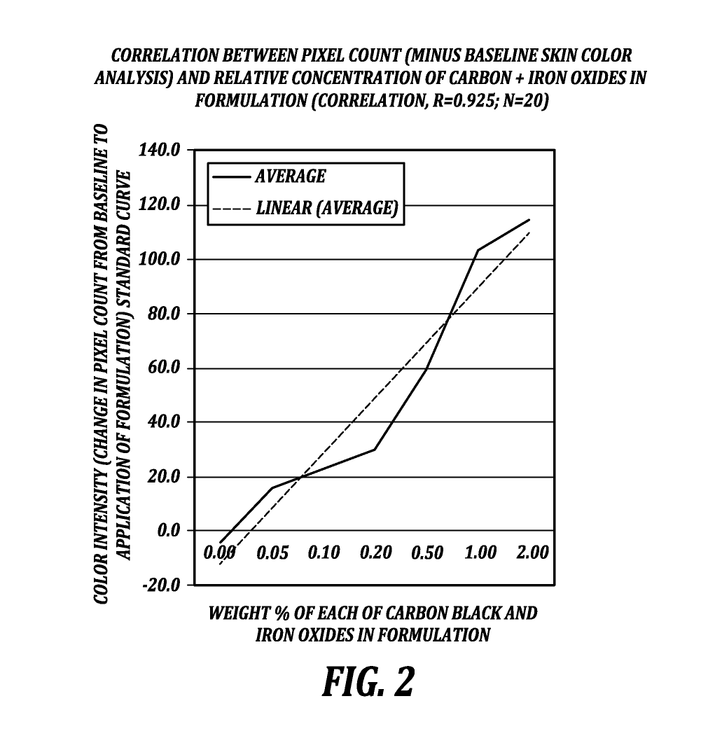

The relationship between carbon black and iron oxide concentration in the formulation was assessed for their correlation to color intensity as determined by image analysis (difference between applied formulation standard curve and baseline skin measurements). In this study an esthetician applied 25 μL of formulation with relative varying levels of particulate pollutants to the volar forearms of 20 study participants. Image analysis demonstrates that color intensity is positively correlated to relative concentration of carbon black and iron oxides in formulation (r=0.925), as illustrated in FIG. 2.

### Exemplary Test Data

Tabulated in Table 1 is image analysis quantification of the relative amount of the Exemplary Formulation remaining on the skin as determined by color intensity (measured in the difference in the white/dark pixels compared to baseline, using a pixel intensity scale of 0=black to 255=white) of the overall average of twenty subjects following manual versus Clarisonic cleansing.

Procedure
i. Twenty subjects were enrolled in a single-center, cleansing study comparison between the sonic skin-cleansing brush compared to manual cleansing.
ii. Equal amount of formulation applied to 32 millimeter circles (1.25 inches) in the center of each cheek (left and right).
iii. Method of cleansing (sonic versus manual) was randomized to the side of the face (left or right) for each subject.
iv. Each side was cleansed for five seconds using the sonic brush head or manually, using equal amounts of water and a gel cleanser.
v. Photographs (VISIA CR, Canfield Imaging, New Jersey, USA) were taken before application of the formulation, after application of the formulation, and following cleansing. Image analysis (Image J, NIH, Bethesda, Md., USA) and color measurements (Dermaspectrometer II) were used to quantify color intensity (amount of formulation).

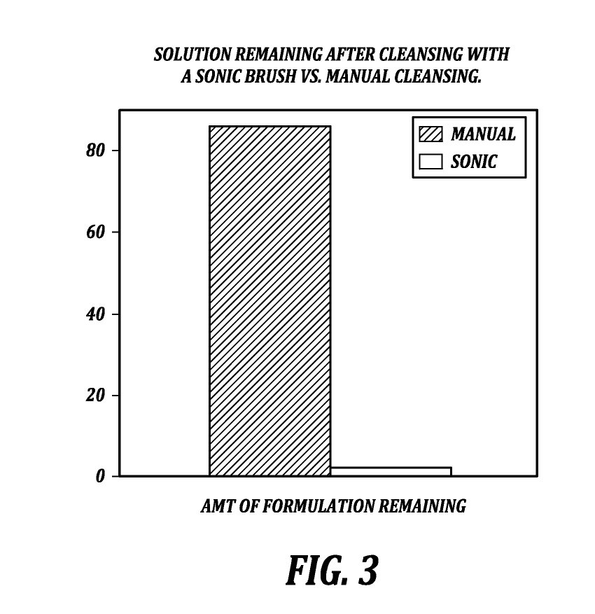

The results indicate that using a robust cleansing protocol to assess removal of formulation indicates that the sonic brush removes 35.8 times more formulation than manual cleansing, as illustrated in TABLE 1 and FIG. 3.

TABLE 1
Image analysis data of the relative amount color (measured
in the difference in dark/light pixels compared to baseline)
remaining on the skin of 20 subjects after cleansing with
a sonic brush versus manual cleansing.

| | CLARISONIC | MANUAL |
|---|---|---|
| Subject | BL-post | BL-post |
| Average | 2.41 | 86.22 |
| Std Dev. | 2.33 | 12.39 |
| Paired t-test | | p < 0.001 |

The sonic brush cleansed significantly better (less formulation remaining on the skin) than manual cleansing; manual cleansing left 35.8 times more formulation on the skin than the sonic brush.

While illustrative embodiments have been illustrated and described, it will be appreciated that various changes can be made therein without departing from the spirit and scope of the invention.

The embodiments of the invention in which an exclusive property or privilege is claimed are defined as follows:

1. A formulation configured to simulate contaminated human skin, comprising:
a synthetic sebum composition, comprising:
one or more triglycerides;
one or more fatty acids;
one or more waxes or wax esters;
one or more hydrocarbons; and
cholesterol or a cholesterol ester; and
a plurality of particles, wherein the plurality of particles has a mean diameter of 10 microns or less.

2. The formulation of claim 1, wherein the one or more triglycerides are selected from the group consisting of glyceryl trioleate, triglyceride-rich oils (e.g., olive oil, cotton seed oil, coconut oil), and combinations thereof.

3. The formulation of claim 1, wherein the one or more fatty acids are selected from the group consisting of oleic acid, palmitic acid, palmitoleic acid, and combinations thereof.

4. The formulation of claim 1, wherein the one or more waxes or wax esters are selected from the group consisting of cetyl palmitate, paraffin wax, spermaceti wax, beeswax, jojoba oil, lanolin, and combinations thereof.

5. The formulation of claim 1, wherein the one or more hydrocarbons are selected from the group consisting of squalene, squalane, petrolatum, mineral oil, and combinations thereof.

6. The formulation of claim 1, further comprising an additive selected from the group consisting of an emulsifier (e.g., polyglyceryl oleate), mineral oil, and petrolatum.

7. The formulation of claim 1, wherein the plurality of particles is selected from the group consisting of carbon black particles, iron oxide particles, and combinations thereof.

8. The formulation of claim 1, wherein the plurality of particles comprises carbon black particles having a mean diameter of about 500 nanometers or less and iron oxide particles having a mean diameter of about 0.3 to about 5 microns.

9. A method of evaluating the efficacy of a skin-cleansing procedure, comprising:
applying a formulation according to claim 1 to a test area of a subject's skin;
performing a skin-cleansing procedure on the test area;
measuring the color of the test area to provide a post-clean color signal; and
comparing the post-clean color signal to a baseline color signal obtained without the formulation.

10. The method of claim 9, wherein the baseline color signal is obtained in the test area prior to the step of applying the formulation.

11. The method of claim 9, wherein the step of measuring the color of the test area comprises digital photograph analysis.

12. The method of claim 9, wherein the test area is on the subject's face or forearm.

13. The method of claim 9, wherein the skin-cleansing procedure is selected from the group consisting of manual cleansing, cleansing with a rotating brush, and cleansing with an oscillating brush.

14. The method of claim 9, wherein the formulation is applied to the test area in an amount of about 1 to about 10 mg/cm².

15. A formulation configured to simulate contaminated human skin, comprising:
a synthetic sebum composition, comprising:
cholesterol;
squalane;
oleic acid;
glyceryl trioleate; and
cetyl palmitate; and
a plurality of particles, wherein the plurality of particles has a mean diameter of 10 microns or less.

16. The formulation of claim 15, wherein the plurality of particles comprises carbon black particles having a mean diameter of about 500 nanometers or less and iron oxide particles having a mean diameter of about 0.3 to about 5 microns.
